(19) United States
(12) Patent Application Publication
(10) Pub. No.: US 2023/0165916 A1
(43) Pub. Date: Jun. 1, 2023

(54) COMPOSITION FOR PREVENTING OR TREATING INFLAMMATORY DISEASES OF DIGESTIVE SYSTEM OR COLITIS, COMPRISING REOVIRUS AS ACTIVE INGREDIENT

(71) Applicant: VIROCURE, INC., Seoul (KR)

(72) Inventors: Yeon Sook LEE, Gunpo-si, Gyeonggi-do (KR); Haeng Jun YOO, Seoul (KR); Sang Kyoung HAN, Seoul (KR)

(73) Assignee: VIROCURE, INC., Seoul (KR)

(21) Appl. No.: 17/995,135

(22) PCT Filed: Jan. 29, 2021
(86) PCT No.: PCT/KR2021/001188

§ 371 Date: Sep. 30, 2022

(30) Foreign Application Priority Data
Jan. 31, 2020 (KR) 10-2020-0012090

(51) Int. Cl. A61K 35/765; A61P 1/04; A23L 33/10

(52) U.S. Cl. CPC A61K 35/765; A61P 1/04; A23L 33/10

(57) ABSTRACT

The present disclosure relates to a composition for preventing, alleviating, or treating an inflammatory disease of the digestive system, particularly, colitis, comprising a reovirus or a variant thereof as an active ingredient.

### TECHNICAL FIELD

The present disclosure relates to a composition for preventing, alleviating, or treating an inflammatory disease of the digestive system, or colitis, including a reovirus as an active ingredient.

### BACKGROUND ART

Since a virus cannot metabolize substances by itself, it propagates by a method of infiltrating its own DNA or RNA into a host cell and then using cell organelles within the host cell to replicate a viral genetic material and produce a virus. The aforementioned process of proliferation may damage or destroy host cells and cause a disease in the host.

However, research that uses the virus for cancer treatment and the like by reversely utilizing such characteristics has been conducted. That is, a principle in which viruses can effectively kill only cancer cells with their replicative infectivity is applied. It is an oncolytic virus, and a wild-type or attenuated virus is used as it is, or may be used to treat cancer by inserting a gene while maintaining infectivity.

A wild-type oncolytic virus is a virus with intrinsically potent oncolytic ability, and is known to induce the destruction of tumor cells with deficient or weakened intracellular antiviral immunity through specific infection and proliferation of these cells. Research on the tumor-specific killing mechanism by the wild-type oncolytic virus has been extensively conducted, and typical examples thereof include research on the development of cancer therapeutic agents using a wild-type reovirus.

Respiratory enteric orphan virus (Reovirus) is a non-enveloped virus whose entire genome consists of 10 double-stranded RNA segments, and is an asymptomatic virus that is ubiquitous in the general environment and does not show symptoms in hosts with normal immune function. It is known that reovirus is weakly infectious to humans and the site of infection is limited to the upper respiratory tract and gastrointestinal tract (Tyler, In: Fields Virology, Knipe and Howeley (Eds.), Lippincott Williams & Wikens, Philadelphia, 1729-1745, 2001).

The reovirus may exhibit potent cytocidal activity when infecting certain types of transformed cells. While research on the tumor-destructive activity of the reovirus has been conducted, it has been reported that cells including activated Ras-signaling pathways that induce tumors are susceptible to reovirus infection in vitro and in vivo (Coffey et al., Science, 282:1332-1334, 1998), and reovirus therapy for cancer patients with no limiting immune system damage is currently in clinical trials (Norman et al., Drug Discov. Today, 10:847-855, 2005).

Meanwhile, colitis is a disease in which erosions or ulcers are continuously formed in the mucosa of the large intestine, bloody stool, mucous stool, diarrhea, and abdominal pain occur, and in severe cases, systemic symptoms such as fever, body weight loss, and anemia appear. Further, Crohn's disease is a disease in which lesions such as ulcers occur discontinuously in any part of the digestive tract from the mouth to the anus, symptoms such as abdominal pain, diarrhea, and bloody stool occur, and in severe cases, symptoms such as fever, melena, body weight loss, general malaise, and anemia occur. Both ulcerative colitis and Crohn's disease are chronic intractable diseases in which temporary remission and recurrence of symptoms get better, and recurrence is repeated, and are commonly called inflammatory bowel diseases.

As existing drugs used to treat the inflammatory bowel disease, steroidal immunosuppressants and 5-aminosalicylic acid (5-ASA)-based drugs which block the production of prostaglandins (for example, sulfasalazine, and the like), mesalazine, and the like are used, and since these drugs not only have little therapeutic effect on inflammatory bowel disease, but also induce serious side effects such as abdominal fullness, headaches, rashes, liver disease, leukopenia, agranulocytosis, and male infertility, the use of the therapeutic agents is limited. Therefore, there is a need for the development of a therapeutic agent for an inflammatory disease of the digestive system, particularly, ulcerative colitis, Crohn's disease and the like, which can be safely used or taken for a long period of time, is highly effective and safe, and does not cause side effects.

Against this background, the present inventors have surprisingly confirmed that a reovirus, which has been mainly developed for anticancer use due to their oncolytic activity in the related art, exhibited excellent anti-inflammatory activity in animal models of colitis without long-term side effects, thereby completing the invention disclosed in the present specification.

### DISCLOSURE

### Technical Problem

One object of the present disclosure is to provide a pharmaceutical composition for preventing or treating colitis, or an inflammatory disease of the digestive system from the oral cavity to the anus, which includes, particularly, the large intestine, including a wild-type reovirus or a variant thereof as an active ingredient.

Another object of the present disclosure is to provide a method for treating colitis, the method including: administering a pharmaceutical composition including a wild-type reovirus or a variant thereof as an active ingredient to a subject having or suffering from an inflammatory disease of the digestive system from the oral cavity to the anus, which includes, particularly, the large intestine.

Still another object of the present disclosure is to provide a health functional food composition for preventing or ameliorating an inflammatory disease of the digestive system from the oral cavity to the anus, which includes, particularly, the large intestine, including a wild-type reovirus or a variant thereof as an active ingredient.

Yet another object of the present disclosure relates to a use of a wild-type reovirus or a variant thereof for preparing a pharmaceutical composition suitable particularly for oral administration in order to treat colitis or an inflammatory disease of the digestive system from the oral cavity to the anus, which includes, particularly, the large intestine.

Yet another object of the present disclosure relates to a use of a wild-type reovirus or a variant thereof for preparing a health functional food composition for preventing, ameliorating, or alleviating colitis or an inflammatory disease of the digestive system from the oral cavity to the anus, which includes, particularly, the large intestine.

### Technical Solution

Hereinafter, each aspect and embodiment described in the present specification is also applicable to each different aspect and embodiment. That is, all combinations of various elements disclosed in the present specification fall within the scope of the present disclosure. Further, the scope of the present application cannot be said to be limited by the specific description described below.

In addition, those skilled in the art can recognize or confirm a plurality of equivalents to the specific aspects of the present disclosure described in the present specification using only routine experimentation. Furthermore, such equivalents are intended to be included in the present disclosure. It will be described in detail as follows.

In the present specification, ranges are referred to briefly so as to avoid having to indicate redundantly and describe each and every value within the range. Any suitable numerical value within the range can be selected as the upper, lower, or end value of the range. For example, the range of 0.1-1.0 (0.1 to 1) indicates the end values of 0.1 and 1.0, as well as the median values of 0.2, 0.3, 0.4, 0.5, 0.6, 0.7, 0.8, and 0.9 and all intermediate ranges encompassed within the range of 0.1-1.0, for example, 0.2-0.5, 0.2-0.8, 0.7-1.0, and the like.

The term “more than 5% and up to 100%” or “more than 5% and up to 100%” refers to herein a range starting at values strictly starting from above 5% and ending at 100%. That is, the term includes all intermediate values from 5 to 100%, but does not include a lower limit of 5%.

Throughout the present specification, unless the context clearly dictates otherwise, the singular form of a word includes the plural form and vice versa. Therefore, singular expressions generally include the plural of each term. For example, when referring to “a method” or “a composition,” a plurality of such “methods” or “compositions” are included. The words “comprise,” “comprises” and “comprising” are to be interpreted inclusively. Likewise, the terms “include,” “including” and “or” are to be interpreted inclusively. However, all these terms are to be interpreted to encompass exclusive embodiments that may be exhibited using words such as “consisting of”.

The term “about” as used herein when referring to measurable values, such as amounts, temperatures, and temporal periods, refers to including a difference of ±10 wt %, more preferably ±5 wt %, and even more preferably ±3%, and the reason is because such a difference is adequate to reproduce the disclosed methods and compositions.

One aspect of the present disclosure to achieve the aforementioned objects provides a pharmaceutical composition for preventing or treating colitis, including a wild-type reovirus or a variant thereof as an active ingredient.

As described herein, the “reovirus” is a non-enveloped virus whose entire genome consists of 10 double-stranded RNA segments, and is an asymptomatic virus that is ubiquitous in the general environment and does not show symptoms in hosts with normal immune function.

As described herein, the reovirus may be wild-type virus, or a naturally or non-naturally occurring variant, but is not limited thereto. The reovirus may be derived from any reovirus, may be a member of the Reoviridae family which may be obtained from various sources, and may be Serotype 1 (Lang), Serotype 2 (Jones) or Serotype 3 (Dearing and Aneny) reovirus or a variant thereof, for example, the RC402 strain (attenuated reovirus in US 2009-0104162 A1 (published date: Apr. 23, 2009)), the RP116 strain (attenuated reovirus described in US 2009-0214479 A1 (published date: Aug. 27, 2009), or derivatives, variants, progenies, or mixtures thereof, and the like, but is not limited thereto.

Further, the reovirus may be derived from one or more reoviruses showing tropism for cells of primates other than humans (chimpanzees, gorillas, macaques, monkeys, and the like), rodents (mice, rats, garyville mice, hamsters, rabbits, guinea pigs, and the like), and other mammals including dogs, cats, and general livestock (cows, horses, pigs, and goats), but is not limited thereto.

As an example of the reovirus variant according to the present disclosure, “attenuated reovirus” may be derived according to other methodologies including the production and identification of sigma 1-deficient and/or sigma 1-defective mutants by molecular biological approaches (and in a specific exemplary embodiment, additionally or alternatively, also including the production and identification of sigma 3-deficient and/or sigma 3-defective mutants) or including the isolation of naturally occurring sigma 1-deficient and/or sigma 1-defective mutants and/or sigma-3 mutants, and/or the artificial induction of such sigma 1 (and/or sigma 3) mutants by chemical, physical and/or genetic techniques (for example, selective recombination of reovirus genes in productively infected host cells).

As described herein, the attenuated reovirus includes an infectious, replication-competent reovirus virion lacking the wild-type reovirus S1 gene and consequently lacking a detectable reovirus sigma 1 capsid protein (that is, viral particles including the viral genome, core protein and protein envelope).

In a specific exemplary embodiment, the attenuated reovirus lacks the wild-type reovirus S4 gene and expresses a mutated reovirus sigma 3 capsid protein. As is known in the relevant art, an infectious, replication-competent reovirus is one that is capable of, upon introduction into a suitable host cell under appropriate conditions and for a sufficient time, binding to and being internalized by the host cell and thus directing replication of the reoviral genome and biosynthesis of reoviral structural proteins in a manner that permits assembly of complete progeny reoviruses that, upon release from the host cell, are capable of productively infecting other host cells to perpetuate the viral replication cycle.

Attenuated reoviruses are described, for example, in US Application Publication Nos. US2009/0214479 and US2009/0104162, each of which is incorporated herein by reference in its entirety.

As an example of the reovirus variant according to the present disclosure, an “inactivated reovirus” refers to a virus whose harmful activity such as infectivity, toxicity or cytotoxicity due to physical or chemical treatment, or genetic manipulation is weakened, reduced, suppressed or eliminated partially, for example, by at least about 10%, at least about 15%, at least about 20%, at least about 25%, at least about 30%, at least about 35%, at least about 40%, at least about 45%, at least about 50% or more, or substantially all (including up to 100%), and simultaneously, beneficial activity, that is, anti-inflammatory activity is enhanced, increased, improved or strengthened by at least about 5%, at least about 10%, at least about 15%, at least about 20%, at least about 25%, at least about 30%, at least about 35%, at least about 40%, at least about 45%, at least about 50% or more, or substantially up to 100% compared to a wild-type or non-engineered-reovirus.

In certain exemplary embodiments, the inactivated reovirus may be a reovirus that has been inactivated through particularly heat treatment. Any heat treatment may be included in the heat treatment according to the present disclosure as long as the heat treatment can enhance, increase, improve or strengthen anti-inflammatory activity compared to a wild-type or untreated reovirus, and it is preferred that reovirus is treated particularly at about 60° C. (±10 to 5° C.) for about 20 minutes (±10 to 5 minutes).

As described herein, the “inactivated reovirus” may be referred to interchangeably with “attenuated reovirus”.

As described herein, “colitis” is a disease in which inflammation occurs in the large intestine due to various causes, and is broadly classified into infectious enteritis and non-infectious enteritis depending on the cause. Acute infectious colitis frequently occurs worldwide, and its main symptoms are fever, nausea, emesis, mucous or bloody diarrhea, and abdominal pain. There are viral enteritis (norovirus and rotavirus), bacterial enteritis (cholera, Escherichia coli, dysentery, typhoid, Yersinia, and campylobacter), protozoan colitis (amoeba), and pseudomembranous colitis depending on the type of infectious bacteria. Inherently, a large number of bacteria are mixed in the intestines, and the intestinal mucosa and the microflora coexist while maintaining a certain ecosystem. However, when highly pathogenic bacteria enter the intestines, the bacteria directly invade the intestinal mucosa, or toxins they produce disrupt this balance, causing bacterial enteritis. Mycobacterium tuberculosis also causes colitis, and chronic abdominal pain or body weight loss frequently occurs. Examples of non-infectious colitis include inflammatory bowel disease (Crohn's disease and ulcerative colitis), radiation colitis, ischemic colitis, Behcet's colitis, drug-induced colitis, and the like.

As used herein, “inflammatory disease of the digestive system” refers to an acute or chronic inflammatory disease that may occur in any part of the digestive tract or system from the oral cavity to the anus, and includes, but is not limited to, an inflammatory bowel disease, for example, esophagitis (including reflux or non-reflux), gastritis (including reflux), duodenitis, enteritis, colitis, and the like.

The “inflammatory bowel disease (IBD)” refers to a disease that induces chronic inflammation in the gastrointestinal tract while being accompanied by symptoms such as abdominal pain, fever, diarrhea and melena. The inflammatory bowel diseases are classified into two forms of ulcerative colitis (UC) and Crohn's disease (CD), ulcerative colitis is a type of diffuse nonspecific inflammation of the large intestine of unknown cause, which usually invades the mucosa to often form sores or ulcers, and is accompanied by various systemic symptoms including bloody diarrhea, and Crohn's disease discontinuously occurs in the entire digestive tract from the oral cavity to the anus, is a granulomatous inflammatory lesion of unknown cause in which an ulcer, fibrosis, stenosis and a lesion progress in the entire enteric layer in the mucosa, and is accompanied by systemic symptoms such as abdominal pain, chronic diarrhea, fever and malnutrition.

Further, as described herein, the “ischemic colitis” is a disease that causes bloody stool and abdominal pain in the elderly, or patients with hypertension and heart disease, and is caused by a reduction in blood flow to the large intestine. The “Behcet's enteritis” is a disease in which ulcers develop in the small intestine or large intestine in patients with Behcet's disease, and recurrence is common, and the “radiation enteritis” is a disease that occurs when the abdomen is subjected to radiation therapy due to a malignant tumor, and abdominal pain or bleeding is common. The “drug-induced enteritis” is a disease that occurs in relation to taking drugs, and anti-inflammatory analgesics are the most common drugs.

In regard to the present disclosure, the colitis is a disease in which inflammation occurs in the large intestine, and may be one or more selected from the group consisting of acute colitis, bacterial colitis, viral colitis, pseudomembranous colitis, inflammatory bowel disease, chronic colitis, ulcerative colitis, Crohn's disease, ischemic colitis, Behcet's colitis, drug-induced colitis, collagenous colitis, lymphocytic colitis, and radiation colitis, but is not limited thereto.

The pharmaceutical composition including the reovirus according to the present disclosure as an active ingredient may prevent or treat colitis.

As described herein, “prevention” refers to suppressing or delaying an inflammatory disease of the digestive system from the oral cavity to the anus or colitis or the onset thereof by administration of the pharmaceutical composition.

As described herein, “treatment” refers to all actions of clinical intervention to alter the natural processes of an individual or cell to be treated, and may be performed usually for the purpose of partially or completely eliminating a clinical pathological condition while it progresses, or for a prophylactic purpose before such a condition develops. The desired therapeutic effect includes preventing the occurrence or recurrence of a disease, alleviating symptoms, reducing all direct or indirect pathological consequences of the disease, preventing metastasis, reducing a disease progression rate, mitigating or temporarily alleviating a condition, making a patient feel better, or improving the prognosis. For the purpose of the present disclosure, the treatment may be interpreted to include all actions in which the symptoms of colitis are ameliorated or completely cured by administering the pharmaceutical composition, but is not limited thereto.

As described herein, the “pharmaceutical composition for preventing or treating colitis or an inflammatory disease of the digestive system” may be prepared into a pharmaceutical formulation using methods well known in the art so as to provide rapid, sustained or delayed release of an active ingredient after being administered to a mammal. In the preparation of the formulation, it is desirable to mix or dilute the active ingredient with a carrier, or enclose the active ingredient in a container-shaped carrier.

The pharmaceutical composition according to the present disclosure may include one or more stabilizers such as a compound which promotes engraftment (for example, an anti-T cell receptor antibody, an immunosuppressant, and the like), albumin, dextran 40, gelatin, and hydroxyethyl starch.

The dosage form of the pharmaceutical composition for preventing or treating colitis of the present disclosure may be used alone or in combination with other pharmaceutically active compounds, as well as in suitable sets.

Therefore, the pharmaceutical composition for preventing or treating colitis of the present disclosure may be used by being formulated into a form such as an oral dosage form such as a powder, a granule, a tablet, a capsule, a suspension, an emulsion, a syrup, and an aerosol, an external preparation, a suppository, and a sterile injection solution, and may further include appropriate carriers, excipients, and diluents typically used in the preparation of the composition.

For example, a carrier, an excipient and a diluent which may be included in the pharmaceutical composition according to the present disclosure may be lactose, dextrose, sucrose, sorbitol, mannitol, xylitol, erythritol, maltitol, starch, acacia rubber, alginate, gelatin, calcium phosphate, calcium silicate, cellulose, methyl cellulose, microcrystalline cellulose, polyvinyl pyrrolidone, water, methyl hydroxybenzoate, propyl hydroxybenzoate, talc, magnesium stearate, and mineral oil, but is not limited thereto. When the pharmaceutical composition is prepared, the pharmaceutical composition is prepared using a diluent or excipient, such as a filler, an extender, a binder, a wetting agent, a disintegrant, and a surfactant, which are commonly used.

The excipient may be in a wide variety of forms depending on the form of preparation desired for administration. For example, an excipient suitable for use in the administration form of an oral liquid or aerosol includes water, a glycol, an oil, an alcohol, a flavoring ingredient, a preservative, and a coloring ingredient, but is not limited thereto. Examples of a preservative suitable for use in a solid oral dosage form (for example, a powder, a tablet, a capsule and a caplet) include starch, sugar, microcrystalline cellulose, an excipient, a granulating agent, a lubricant, a binder and a disintegrant, but are not limited thereto. Because of their convenience in administration, tablets and capsules are the most useful oral dosage unit forms, and in this case, a solid excipient is applied. Preferably, the tablet may be coated using standard aqueous or non-aqueous techniques. Examples of an excipient which may be used in the oral dosage form of the present disclosure may include a binder, a filler, a disintegrant, and a lubricant. Binders suitable for use in pharmaceutical compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (for example, ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

Disintegrants that can be used in pharmaceutical compositions according to the present disclosure are used to disintegrate tablets when the tablets are exposed to an aqueous environment. Tablets including excessively large amounts of disintegrants may be disintegrated during storage, whereas tablets including excessively small amounts of disintegrants are not disintegrated at a preferred rate under preferred conditions. Therefore, a sufficient amount of disintegrant, neither too large nor too small, needs to be used to form the solid oral dosage form of the present disclosure so as to be suitable for regulating the release of the active ingredient. The amount of disintegrant used varies depending on the type of preparation and may be readily determined by those with ordinary skill in the art to which the present disclosure pertains. Typically, the tablet may include about 0.5 to about 15 weight percent, preferably about 1 to about 5 weight percent of disintegrant.

Lubricants that can be used in the pharmaceutical composition according to the present disclosure may include calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oils (for example, peanut oil, cottonseed oil, sunflower oil, sesame oil, olive oil, corn oil, and soybean oil), zinc stearate, ethyl oleate, ethyl laureate, agar and mixtures thereof, but are not limited thereto.

The pharmaceutical composition according to the present disclosure may include one or more compounds capable of reducing the rate at which the active ingredient, that is, the reovirus, is destroyed or inactivated in the body. The compound may include a stabilizer, an antioxidant such as ascorbic acid, a pH buffer, or a salt buffer, but is not limited thereto.

A solid preparation for oral administration includes a tablet, a pill, a powder, a granule, a capsule, and the like, and the solid preparation is prepared by mixing at least one excipient, for example, starch, calcium carbonate, sucrose or lactose, gelatin, and the like with the compound. Further, in addition to simple excipients, lubricants such as magnesium stearate and talc may also be used.

A liquid preparation for oral administration corresponds to a suspension, a liquid for internal use, an emulsion, a syrup, and the like, and the liquid formulation may include, in addition to water and liquid paraffin which are simple commonly used diluents, various excipients, for example, a wetting agent, a sweetener, an aromatic, a preservative, and the like.

Examples of a preparation for parenteral administration include an aqueous sterile solution, a non-aqueous solvent, a suspension, an emulsion, a freeze-dried preparation, and a suppository. As the non-aqueous solvent and the suspension, it is possible to use propylene glycol, polyethylene glycol, a vegetable oil such as olive oil, an injectable ester such as ethyl oleate, and the like. As a base of the suppository, it is possible to use Witepsol, Macrogol, Tween 61, cacao butter, laurin fat, glycerogelatin, and the like.

The dosage of the composition for preventing or treating an inflammatory disease of the digestive system from the oral cavity to the anus or colitis of the present disclosure varies depending on the patient's condition and body weight, degree of disease, drug form, administration route and duration, but may be appropriately selected by those skilled in the art. The administration may be carried out once daily, or may be divided into several times. The dosage is not intended to limit the scope of the present disclosure in any way. An effective amount is the amount of compound required to give the desired effect. Effective amounts may vary depending on the route of administration, the use of excipients, and the possibility of use with other agents, as will be appreciated by those skilled in the art.

The effective amount of reovirus of the present disclosure is a dose required for a sufficient time to alleviate, improve, mitigate, ameliorate, and stabilize a disease, suppress the spread of the disease, slow or delay the progression of the disease, and heal the disease.

The effective amount may vary depending on a number of factors such as the pharmacokinetic characteristics of the virus, the method of administration, a patient's age, health status and body weight, the nature and extent of the disease state, the number of treatments and the current mode of treatment, and may also vary depending on, for example, the virulence and titer of the virus. Those skilled in the art may adjust the appropriate amount based on the above factors. The virus initially may be administered in an appropriate amount as needed, depending on the patient's clinical response. The effective amount of virus may be empirically determined and may be determined by the maximum amount of virus that can be safely administered and the minimum amount of virus that induces the desired result.

When the reovirus of the present disclosure is systemically administered, a considerably high dose of reovirus may be required to be administered in order to induce clinical effects similar to those achieved by injecting the reovirus into a disease site. However, an appropriate dose level should be the minimum amount that achieves the desired results.

The concentration of reovirus administered may vary depending on the characteristics of the target cells. The administered reovirus concentration may be about 10⁶ to 10¹⁰. As a specific exemplary embodiment of the present disclosure, an amount of about 10⁷ to 10¹⁰ plaque forming units (“pfu”) may be administered in a single dose to a subject, for example, a subject having or suffering from an inflammatory disease of the digestive system from the oral cavity to the anus or colitis, particularly, a human or a non-human mammal.

An effective amount of reovirus may be administered repeatedly depending on the effect of the initial therapeutic regimen. Typically, administration is periodically administered while monitoring all responses. Those skilled in the art may readily ascertain that doses lower or higher than those indicated above may be administered depending on the dosing schedule and the selected route.

Another aspect of the present disclosure provides a method for treating an inflammatory disease of the digestive system from the oral cavity to the anus or colitis, the method including: administering a pharmaceutical composition including a wild-type reovirus or a variant thereof as an active ingredient to a subject having or suffering from an inflammatory disease of the digestive system from the oral cavity to the anus or colitis.

As described herein, the terms “reovirus,” “colitis,” “inflammatory disease of the digestive system,” and “treatment” are as described above.

As described herein, “subject” may refer to all animals including humans. The animal may be not only a human but also a mammal such as a cow, a horse, a sheep, a pig, a goat, a camel, an antelope, a dog, and a cat in need of treatment or amelioration of similar symptoms. Further, the animal may refer to an animal other than a human, but is not limited thereto.

As described herein, “administration” refers to introduction of the composition of the present disclosure to a subject in need by any appropriate method, and for the route of administration of the composition of the present disclosure, the composition of the present disclosure may be administered via various routes of oral or parenteral administration, as long as it may reach a target tissue.

As a route of administration of the pharmaceutical composition, the pharmaceutical composition may be administered via any general route as long as the pharmaceutical composition can reach a target tissue. The pharmaceutical composition of the present disclosure may be administered via a route such as intraperitoneal administration, intravenous administration, intramuscular administration, subcutaneous administration, intradermal administration, oral administration, intranasal administration, intrapulmonary administration, or intrarectal administration, depending on the purpose, but is not particularly limited thereto. However, since the composition may be denatured by gastric acid when administered orally, the composition for oral administration may be formulated to coat an active agent or to protect it from degradation in the stomach. Further, the composition may be administered by any device such that an active material may be transported to a target cell.

Still another aspect of the present disclosure provides a health functional food composition for preventing or ameliorating an inflammatory disease of the digestive system from the oral cavity to the anus or colitis, including a wild-type reovirus or a variant thereof as an active ingredient.

As described herein, the terms “reovirus,” “colitis” and “prevention” are as described above.

Since the health functional food composition of the present disclosure can be ingested on a daily basis, it can be expected to have an effect of preventing or ameliorating colitis, and thus is very useful.

As described herein, “amelioration” refers to all actions that at least reduce a parameter associated with a condition to be treated by administration of the composition, for example, the degree of symptoms.

The health functional food according to the present disclosure can be prepared by a method typically used in the art, and may be prepared by adding raw materials and components typically added in the art during preparation. In addition, the preparation of the health functional food can also be prepared without limitation as long as it is recognized as a food. The health functional food composition of the present disclosure may be prepared in various forms of formulations, and unlike general drugs, has advantages in which there are no side effects, which may occur during long-term administration of drugs, by employing food as a raw material, unlike general drugs, can be ingested on a daily basis due to excellent portability, so that the health functional food composition of the present disclosure is very useful and can be ingested as an adjuvant for enhancing the therapeutic or preventive effect on an inflammatory bowel disease, and the like.

The health functional food is an essential ingredient, has no particular limitation on ingredients other than the reovirus, and may contain various herbal extracts, food supplements, natural carbohydrates, and the like as additional ingredients like typical health functional foods. Furthermore, the food supplement may include food supplements typically used in the art, for example, a flavoring agent, a savoring agent, a colorant, a filler, a stabilizer, and the like, but is not limited thereto.

Examples of the natural carbohydrate include typical sugars such as monosaccharides, for example, glucose, fructose and the like; disaccharides, for example, maltose, sucrose and the like; and polysaccharides, for example, dextrin, cyclodextrin and the like, and sugar alcohols such as xylitol, sorbitol, and erythritol. As the flavoring agent other than those described above, a natural flavoring agent (for example, rebaudioside A, glycyrrhizin and the like) and a synthetic flavoring agent (saccharin, aspartame and the like) may be advantageously used.

In addition to the aforementioned components, the health functional food composition of the present disclosure may contain various nutrients, vitamins, minerals (electrolytes), flavoring agents such as synthetic flavoring agents and natural flavoring agents, colorants, fillers (cheese, chocolate, and the like), pectic acid and salts thereof, alginic acid and salts thereof, organic acids, protective colloid thickeners, pH adjusting agents, stabilizers, preservatives, glycerin, alcohols, carbonating agents used in a carbonated beverage, or the like, and may additionally contain pulp for producing natural fruit juices, fruit juice beverages and vegetable beverages. These ingredients may be used either alone or in combinations thereof. Further, the health functional food may be in the form of any one of meats, sausages, breads, chocolates, candies, snacks, confectioneries, pizzas, ramens, gums, ice creams, soups, beverages, teas, functional water, drinks, alcoholic beverages and vitamin complexes.

In addition, the health functional food may additionally include food additives, and unless otherwise stipulated, whether or not an item is suitable as a “food additive” is determined according to the specifications and criteria for the item in question, in accordance with the General Rules and General Test Methods for Food Additives Code, and the like approved by the Ministry of Food and Drug Safety. In this case, the content of the composition added to foods including beverages in the process of manufacturing a health functional food may be appropriately added or reduced according to the requirements.

### Advantageous Effects

Since it was confirmed that a composition including the reovirus of the present disclosure as an active ingredient exhibits an excellent anti-inflammatory effect in an animal model in which colitis is induced using dextran sulfate sodium (DSS), the composition of the present disclosure can be very usefully used for the prevention, treatment or amelioration of colitis.

### DESCRIPTION OF DRAWINGS

FIG. 1 illustrates a series of processes of orally administering a reovirus-containing composition to a DSS-induced animal model.

FIG. 2 illustrates the results of measuring cell viability after heat-inactivation of a wild-type reovirus (RC402).

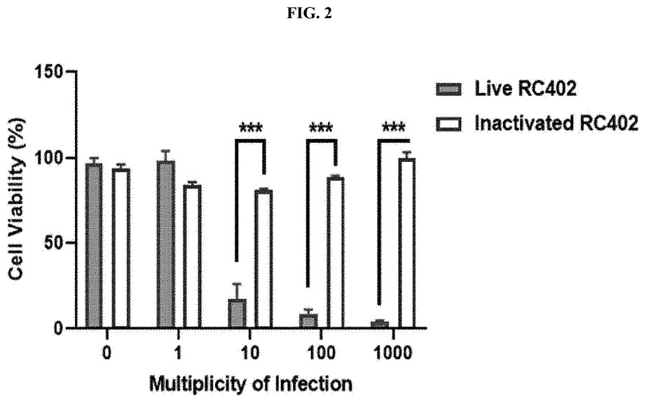

FIG. 3 shows a disease activity index (DAI) reduction effect exhibited by the composition of the present disclosure including a reovirus.

FIG. 4 shows the effect of suppressing the body weight loss in a subject exhibited by the composition of the present disclosure including a reovirus.

FIG. 5 shows the effect of suppressing the decrease in colon length in a subject exhibited by the composition of the present disclosure including a reovirus.

### MODES OF THE INVENTION

Hereinafter, the present disclosure will be described in more detail through examples. These examples are only for describing the present disclosure, and it will be obvious to those skilled in the art that the scope of the present disclosure is not interpreted to be limited by these examples.

### Example 1. Heat-Inactivation of Reovirus

To prepare a reovirus for use in the present disclosure, the reovirus was obtained by heat-inactivating a wild-type reovirus.

Specifically, wild-type reovirus human type 3 (Dearing) was purchased from the American Type Culture Collection (ATCC, VR-824). The reovirus was first isolated from the stool of a child with diarrhea around 1955 and was donated by Dr. Albert Sabin. A BHK21 cell line, HEK293 or L929 cells were inoculated with virus at a multiplicity of infection (MOI) of about 1 to 10 and cultured at 37° C. for 48 to 72 hours to propagate a wild-type reovirus. Thereafter, the cells were freeze-thawed, crushed, and then centrifuged to remove cell debris, and the titer of the liberated reovirus was confirmed using the plaque assay method using a L929 monolayer and the cells were used while being stored at −80° C.

After the obtained wild-type reovirus was heat-inactivated by being stiffed in a water bath at about 60° C. for about 20 minutes, the ability of the virus to kill L929 cells was confirmed using a wst1 assay (Roche kit), and the inactivation efficiency was confirmed through this.

As a result, a heat-inactivated reovirus (referred to as ‘heat-inactivated RC402’) with a titer of 10⁸ PFU/100 μl PBS was obtained.

### Example 2. Construction of DSS-Induced Colitis Animal Model

The animals used in this experiment were 7- to 8-week-old female BALB/C mice and purchased from Orient Bio Co., Ltd., Seoul, Korea. The mice were tested after an adaptation period of 7 days in an animal laboratory of the ViroCure Institute and the supply of water and feed to the mice was not limited during the adaptation period. The experimental animals were provided with a standardized environment, day and night were maintained at 12-hour intervals, and room temperature (23±2° C.) and humidity (50 to 55%) were maintained at appropriate levels.

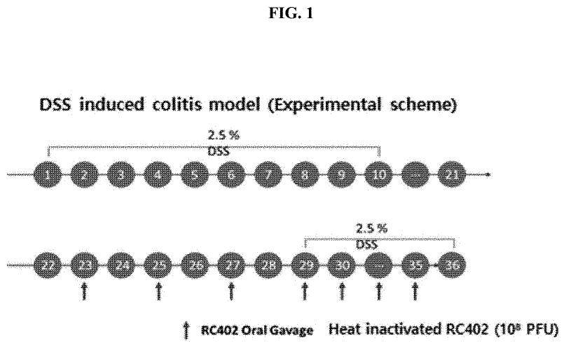

As illustrated in FIG. 1, colitis was induced by changing the normal drinking water of the mice to 2.5% (w/v) dextran sulfate sodium salt (DSS) (MP Biomedicals, Cat #. 9011-18-1) and supplying it for 10 days (1 to 10 days), and thereafter, a recovery period of about 12 days (11 to 12 days) was given to the mice by changing the 2.5% (w/v) DSS to normal drinking water (filtered & autoclaved normal water) and colitis was again induced by re-supplying DSS (2.5%) from approximately day 29.

### Example 3. Oral Administration of Composition Including Reovirus

Experiments were performed by dividing the mice used in this experiment into a normal control, a DSS-induced colitis group, and a reovirus-administered group.

The method of administering a reovirus to the colitis animal model was performed using an oral administration method.

A heat-inactivated reovirus (heat inactivated RC402) was administered orally at about 10⁸ PFU/100 μl PBS at 2-day intervals from approximately day 23 and then administered daily from approximately day 29.

### Example 4. Analysis of Therapeutic Effect of Composition Including Reovirus Using DSS-Induced Colitis Animal Model

From approximately day 29, experiments were performed while observing the presence or absence of colitis and the degree of symptoms by measuring the body weight and observing the stool and anal conditions. On approximately day 36, changes in mouse colon length were measured.

4-1. Visual Evaluation by Measurement of Disease Activity Index (DAI)

In order to measure the colitis intensity of colitis animal model mice treated with oral administration of the composition including the reovirus of Example 3, a disease activity index (DAI) was measured by confirming the changes in body weight, stool consistency, and the presence or absence of bloody stool, which is observed with the naked eye in the stool or anus daily for 7 days according to the disease activity index (DAI) grades in the following Table 1.

TABLE 1

| | | | Presence or |
|---|---|---|---|
| | | | absence of |
| Grade | Reduction in body weight | Stool consistency | bloody stool |
| 0 | No change in body weight | Normal | Normal |
| 1 | 1 to 5% | — | — |
| 2 | 5 to 10% | Loose stool | Occult blood |
| 3 | 10 to 20% | — | — |
| 4 | >20% | Diarrhea | Bloody stool |
| | | | observed with |
| | | | naked eye |

In the DSS-administered mice, loose stool and visually observed bloody stool began to be seen from day 4, and diarrhea and bloody stool could be observed in all mice on day 7.

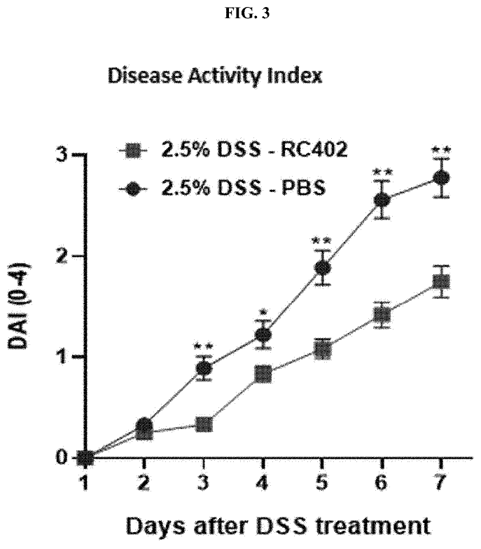

In the reovirus administration group using the oral administration method, diarrhea was relatively reduced and bloody stool was ameliorated, so it was confirmed that the disease activity index was decreased during the administration period compared to the DSS colitis group (FIG. 3).

From this result, it can be seen that not only a wild-type reovirus, but also a reovirus variant, such as a heat-inactivated reovirus, can provide a significant effect in treating and ameliorating colitis and a bowel disease accompanied by inflammation through oral administration, particularly without inducing observable side effects over a long period of time.

4-2. Measurement of Changes in Body Weight and Colon Length

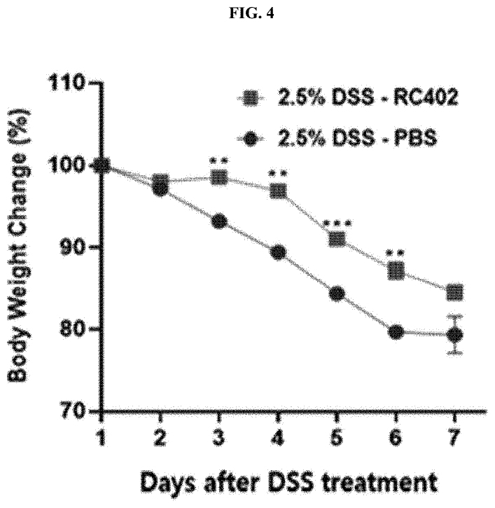

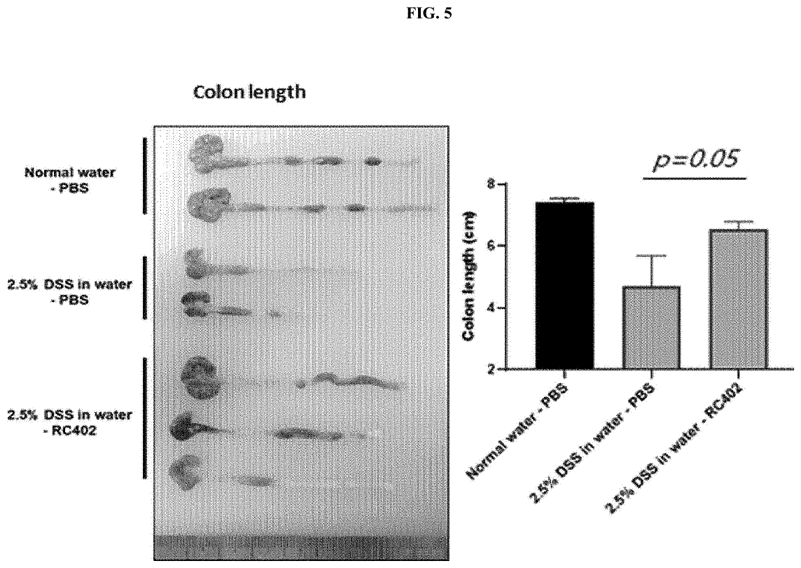

The colitis animal models exhibits symptoms of a decrease in body weight and a decrease in colon length compared to the normal control. As a result of oral administration of a composition including the reovirus of the present disclosure to the colitis animal model, it was confirmed that body weight loss was suppressed and a decrease in colon length was significantly suppressed (FIGS. 4 to 5).

Through this, it can be seen that not only a wild-type reovirus, but also a reovirus variant, such as a heat-inactivated reovirus, can provide a significant effect in treating and ameliorating a disease of the digestive system accompanied by inflammation, including colitis, upon oral administration by suppressing the body weight loss and suppressing the decrease in colon length, particularly without inducing observable side effects over a long period of time.

### Example 5. Statistical Analysis

Statistical analysis was performed using GraphPad Prism 6. Among the One-way ANOVA tests, the Dunnett's multiple comparison test was used to compare differences between the normal control, the DSS-induced colitis group, and the reovirus-administered group. Differences with P-values less than 0.05 were considered statistically significant. Data is presented as mean and SEM.

From the foregoing description, it will be understood by those skilled in the art to which the present disclosure pertains that the present disclosure can be implemented in other concrete forms without modifying the technical spirit or essential features of the present invention. In this regard, it should be understood that the above-described embodiments are only exemplary in all aspects and are not restrictive. The scope of the present disclosure is represented by the claims to be described below rather than the detailed description, and it should be interpreted that the meaning and scope of the claims and all the changes or modified forms derived from the equivalent concepts thereto fall within the scope of the present disclosure.

### INDUSTRIAL APPLICABILITY

A composition including the reovirus according to the present disclosure or a variant thereof is expected to be usefully utilized in treating and ameliorating a disease of the digestive system accompanied by inflammation, including colitis, particularly without inducing observable side effects over a long period of time.

1. A method for preventing, alleviating, or treating colitis, the method comprising administering a composition comprising a wild-type reovirus or a variant thereof as an active ingredient to a subject having or suffering from colitis.

2. The method of claim 1, wherein the wild-type reovirus is Type 1 (Lang), Type 2 (Jones) or Type 3 (Dearing and Abney) wild-type reovirus or a variant thereof.

3. The method of claim 2, wherein the variant is RP116.

4. The method of claim 1, wherein the reovirus variant is a heat-inactivated variant of the wild-type reovirus.

5. The method of claim 1, wherein the colitis is selected from the group consisting of acute colitis, bacterial colitis, viral colitis, pseudomembranous colitis, inflammatory bowel disease, chronic colitis, ulcerative colitis, Crohn's disease, ischemic colitis, Behcet's colitis, drug-induced colitis, collagenous colitis, lymphocytic colitis, and radiation colitis.

6. The method of claim 1, wherein the composition is formulated for oral administration.

7. The method of claim 1, wherein an effective dose of the active ingredient 10⁷ to 10⁹ pfu.

8. The method of claim 1, wherein the composition provides the effects of a decrease in disease activity index (DAI), an increase in body weight or an increase in colon length.

9. (canceled)

10. The method of claim 1, wherein the composition is a pharmaceutical composition or a health functional food composition.
